(19) United States
(12) Patent Application Publication
(10) Pub. No.: US 2025/0236597 A1
(43) Pub. Date: Jul. 24, 2025

(54) DEUTERATED BENZODIAZEPINE ANALOGS AND METHODS OF USE IN TREATING CANCER

(71) Applicants: University of Cincinnati, Cincinnati, OH (US); UWM Research Foundation, Inc., Milwaukee, WI (US)

(72) Inventors: Soma Sengupta, Cincinnati, OH (US); Daniel Pomeranz Krummel, Cincinnati, OH (US); Donatien Kamdem Toukam, Cincinnati, OH (US); James Cook, Whitefish Bay, WI (US); Taukir Ahmed, Milwaukee, WI (US)

(73) Assignees: University of Cincinnati, Cincinnati, OH (US); UWM Research Foundation, Inc., Milwaukee, WI (US)

(21) Appl. No.: 18/855,496

(22) PCT Filed: Apr. 12, 2023
(86) PCT No.: PCT/US2023/018272

§ 371 Date: Oct. 9, 2024

Related U.S. Application Data
(60) Provisional application No. 63/330,051, filed on Apr. 12, 2022.

(51) Int. Cl. C07D 243/24; A61K 31/5513; A61K 45/06

(52) U.S. Cl. CPC C07D 243/24; A61K 31/5513; A61K 45/06

(57) ABSTRACT

Provided herein are benzodiazepine analogs according to the following formula: (I) wherein: R₁ is selected from the group consisting of hydrogen, halo, methyl, ethyl, trideuteromethyl, trifluoromethyl, and cyclopropyl; and R₂ and R₃ are each independently selected from the group consisting of hydrogen, deuterium, tritium, and methyl. Pharmaceutical compositions including Formula I compounds and methods of treating cancer by administering Formula I compounds are also provided herein.

### CROSS REFERENCE TO RELATED APPLICATION

This application claims priority to U.S. Provisional Application Ser. No. 63/330,051, filed Apr. 12, 2022, the entire contents of which are incorporated herein by reference.

### TECHNICAL FIELD

The present disclosure relates to the field of deuterated benzodiazepine analogs and their use in treating cancer.

### BACKGROUND

GABA, or y-aminobutyric acid, is an amino acid that functionally acts as a neurotransmitter and is critical to neurotransmission. Type-A GABA neurotransmitter receptors are a major inhibitory neurotransmitter receptor in the mammalian central nervous system (CNS), but these same receptors are also present outside of the CNS. Genes coding for subunits of Type-A GABA neurotransmitter receptors are expressed in disparate cancer cells and it has been shown that cancer cells possess intrinsic functional Type-A GABA neurotransmitter receptors.

Type-A GABA neurotransmitter receptors are significant pharmacologic targets for the treatment of various neurological disorders, including anxiety and epilepsy. Among the therapeutic agents that work through acting on the Type-A GABA neurotransmitter receptors are the benzodiazepines, which bind at the interface between the alpha and gamma subunits of the pentameric structure (see FIG. 5). Benzodiazepines function to enhance the effectiveness (i.e., chloride anion transport) of GABA, the natural ligand of Type-A GABA neurotransmitter receptors.

A need exists for new cancer therapies that leverage Type-A GABA neurotransmitter receptor function, particularly for the treatment of cancer.

### SUMMARY

Accordingly, provided herein are benzodiazepine analogs that enhance chloride anion efflux in cancer cells, thereby initiating a cascade of events that impairs cancer cell viability.

In one embodiment, a compound according to Formula I is provided, or a pharmaceutically acceptable salt, racemate, or enantiomer thereof:

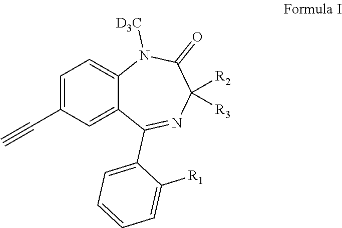

wherein R₁ is selected from the group consisting of hydrogen, halo, methyl, ethyl, trideuteromethyl, trifluoromethyl, and cyclopropyl; and R₂ and R³ are each independently selected from the group consisting of hydrogen, deuterium, tritium, and methyl.

In another embodiment, a pharmaceutical composition is provided, comprising: an effective amount of a compound according to Formula I; and a pharmaceutically acceptable carrier.

In another embodiment, a method of treating cancer in a subject in need thereof is provided, the method comprising administering to the subject an effective amount of a compound according to Formula I, or a pharmaceutically acceptable salt, racemate, or enantiomer thereof.

These and other objects, features, embodiments, and advantages will become apparent to those of ordinary skill in the art from a reading of the following detailed description and the appended claims.

### BRIEF DESCRIPTION OF THE DRAWINGS

The details of embodiments of the presently-disclosed subject matter are set forth in this document. Modifications to embodiments described in this document, and other embodiments, will be evident to those of ordinary skill in the art after a study of the information provided herein.

FIG. 1A is a graph depicting viability of patient-derived melanoma cell line A375 cells treated with DiD3 at varying concentrations for 72 hr.

FIG. 1B is a graph depicting viability of patient-derived glioblastoma cell line LN18 cells treated with DiD3 at varying concentrations for 72 hr.

FIG. 1C is a graph depicting viability of patient-derived lung cancer cell line H1792 cells treated with DiD3 at varying concentrations for 72 hr.

FIG. 1D is a graph depicting viability of A375 cells treated with DiD5 at varying concentrations for 72 hr.

FIG. 1E is a graph depicting viability of LN18 cells treated with DiD5 at varying concentrations for 72 hr.

FIG. 1F is a graph depicting viability of H1792 cells treated with DiD5 at varying concentrations for 72 hr.

FIG. 2A is a graph depicting viability of A375 cells treated with QHD3 at varying concentrations for 72 hr.

FIG. 2B is a graph depicting viability of LN18 cells treated with QHD3 at varying concentrations for 72 hr.

FIG. 2C is a graph depicting viability of H1792 cells treated with QHD3 at varying concentrations for 72 hr.

FIG. 2D is a graph depicting viability of A375 cells treated with QHD5 at varying concentrations for 72 hr.

FIG. 2E is a graph depicting viability of LN18 cells treated with QHD5 at varying concentrations for 72 hr.

FIG. 2F is a graph depicting viability of H1792 cells treated with QHD5 at varying concentrations for 72 hr.

FIG. 3A is a graph depicting viability of A375 cells treated with KRMD3 at varying concentrations for 72 hr.

FIG. 3B is a graph depicting viability of LN18 cells treated with KRMD3 at varying concentrations for 72 hr.

FIG. 3C is a graph depicting viability of H1792 cells treated with KRMD3 at varying concentrations for 72 hr.

FIG. 3D is a graph depicting viability of A375 cells treated with KRMD5 at varying concentrations for 72 hr.

FIG. 3E is a graph depicting viability of LM18 cells treated with KRMD5 at varying concentrations for 72 hr.

FIG. 3F is a graph depicting viability of H1792 cells treated with KRMD5 at varying concentrations for 72 hr.

FIG. 4A is a graph depicting viability of H1792 cells treated with QHD3 at varying concentrations for 72 hr.

FIG. 4B is a graph depicting viability of H1792 cells treated with 20 μM DiD3+QHD3 at varying concentrations for 72 hr.

FIG. 4C is a graph depicting the comparison of the data of FIG. 4A with the data of FIG. 4B.

FIG. 5 depicts the structure and function of Type-A GABA receptors. As shown in the left panel, Type-A GABA receptors are composed most commonly of two α, two β, and γ subunits. Type-A GABA receptors consist of five subunit transmembrane segments that create the chloride (Cl⁻) conduction pore. Inter-subunit binding sites for GABA and benzodiazepine are shown, recognizing the αβαβγ subunit stoichiometry. As shown in the center panel, binding of GABA (agonist) to Type-A GABA receptors leads to Cl⁻ transport. As shown in the right panel, binding of benzodiazepine to Type-A GABA receptors enhances Cl⁻ transport.

FIG. 6 depicts the synthetic scheme for exemplary Formula I compounds according to embodiments of the disclosure.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood in the art, definitions are set forth to facilitate explanation of the presently disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

For the purposes of defining the present technology, the transitional phrase “consisting of” may be introduced in the claims as a closed preamble term limiting the scope of the claims to the recited components or steps and any naturally occurring impurities. For the purposes of defining the present technology, the transitional phrase “consisting essentially of” may be introduced in the claims to limit the scope of one or more claims to the recited elements, components, materials, or method steps as well as any non-recited elements, components, materials, or method steps that do not materially affect the novel characteristics of the claimed subject matter. The transitional phrases “consisting of” and “consisting essentially of” may be interpreted to be subsets of the open-ended transitional phrases, such as “comprising” and “including,” such that any use of an open ended phrase to introduce a recitation of a series of elements, components, materials, or steps should be interpreted to also disclose recitation of the series of elements, components, materials, or steps using the closed terms “consisting of” and “consisting essentially of.” For example, the recitation of a composition “comprising” components A, B, and C should be interpreted as also disclosing a composition “consisting of” components A, B, and C as well as a composition “consisting essentially of” components A, B, and C. Any quantitative value expressed in the present application may be considered to include open-ended embodiments consistent with the transitional phrases “comprising” or “including” as well as closed or partially closed embodiments consistent with the transitional phrases “consisting of” and “consisting essentially of.”

When the term “independently selected” is used, the substituents being referred to (e.g., R groups, such as groups R₂ and R₃), can be identical or different. For example, both R₂ and R₃ can be the same substituent, or R₂ and R₃ can each be different substituents selected from a specified group.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a,” “an,” and “the” include plural references unless the content clearly dictates otherwise.

A “pharmaceutically acceptable salt” is a cationic salt formed at any acidic (e.g., hydroxamic or carboxylic acid) group, or an anionic salt formed at any basic (e.g., amino) group. Many such salts are known in the art, as described in WO 1987/005297, by Johnston et al., published Sep. 11, 1987. Specific cationic salts include the alkali metal salts (such as sodium and potassium), and alkaline earth metal salts (such as magnesium and calcium) and organic salts. Specific anionic salts include halide (such as chloride, bromide, or fluoride salts), sulfate, and maleate. In embodiments, suitable pharmaceutically acceptable salts include, but are not limited to, halide, sodium, sulfate, acetate, phosphate, diphosphate, potassium, maleate, calcium, citrate, mesylate, nitrate, tartrate, aluminum, gluconate, carboxylate, and the like.

Such salts are well understood by the skilled artisan and the skilled artisan is able to prepare any number of salts given the knowledge in the art. Furthermore, it is recognized that the skilled artisan may select one salt over another for reasons of solubility, stability, formulation ease and the like. Determination and optimization of such salts is within the purview of the skilled artisan's practice.

The terms “enantiomer” and “racemate” have the standard art recognized meanings (see, e.g., Hawley's Condensed Chemical Dictionary, 16th ed. (2016)). The illustration of specific protected forms and other derivatives of the compounds of the instant invention is not intended to be limiting. The application of other useful protecting groups, salt forms, esters, and the like is within the purview of the skilled artisan.

The terms “halo” or “halogen,” as used herein, refer to fluoro (F), chloro (Cl), bromo (Br), and iodo (I) groups.

“Alkynyl,” as used herein, refer to a univalent hydrocarbon radical containing a triple bond. In embodiments, alkynyl is represented as-C═C.

“Deuterium” (D), also known as heavy hydrogen or hydrogen-2, refers to an isotope of hydrogen that has one proton and one neutron in its nucleus and has twice the mass of hydrogen. A deuterated compound is a compound to which a deuterium atom has been introduced to replace hydrogen. Trideuteromethyl, or CD₃, is a methyl group wherein the hydrogen atoms have been replaced with deuterium.

“Tritium” (T), also known as hydrogen-3, refers to a radioactive isotope of hydrogen that has one proton and two neutrons. A tritiated compound is a compound to which a tritium atom has been introduced to replace hydrogen.

As used herein, the terms “treatment” or “treating” of a condition and/or a disease in an individual, including a human or lower mammal, means:
(i) preventing the condition or disease, that is, avoiding any clinical symptoms of the disease, particularly in individuals at risk for developing the condition or disease;
(ii) inhibiting the condition or disease, that is, arresting the development or progression of clinical symptoms; and/or
(iii) relieving the condition or disease, that is, causing the regression of clinical symptoms.

The terms “effective amount” or “therapeutically effective amount” as defined herein in relation to the treatment of cancer, refer to an amount that will decrease, reduce, inhibit, or otherwise abrogate the growth of a cancer cell or tumor. The specific therapeutically effective amount will vary with such factors as the particular disease being treated, the physical condition of the individual being treated, the duration of the treatment, the nature of concurrent therapy (if any), and the specific formulations employed.

As used herein, the terms “administer” or “administration” may comprise administration routes such as enteral (e.g., oral, sublingual, buccal, or rectal), parenteral (e.g., intravenous, intramuscular, subcutaneous, intraarterial, intratumoral), intranasal, inhaled, vaginal, transdermal, etc., so long as the route of administration results in an anti-cancer effect in the subject. In specific embodiments, the administration route is oral, intravenous, or intratumoral.

As used herein, the term “subject” generally refers to a living being (e.g., animal or human) capable of suffering from cancer. In a specific embodiment, the subject is a mammal. In a more specific embodiment, the subject is a human subject.

Provided herein are benzodiazepine analogs that enhance chloride anion efflux in cancer cells, thereby initiating a cascade of events that impairs cancer cell viability.

The compounds disclosed herein are analogs of benzodiazepine compounds such as diazepam, QH-II-66, and KRM-II-08, which compounds have the following structures:

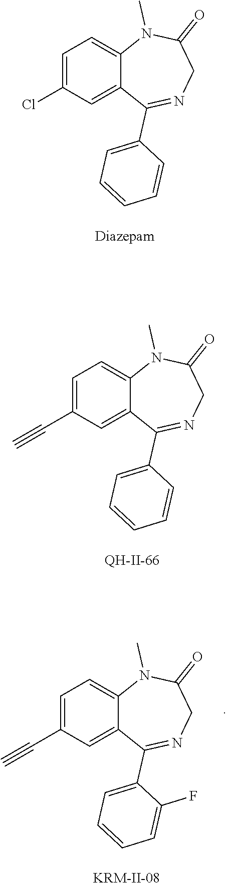 [SMILES: CN1C(=O)CN=C(c2ccccc2)c2cc(Cl)ccc21 ; C#Cc1ccc2c(c1)C(c1ccccc1)=NCC(=O)N2C ; C#Cc1ccc2c(c1)C(c1ccccc1F)=NCC(=O)N2C ; "."]

In one embodiment, a compound according to Formula I is provided, or a pharmaceutically acceptable salt, racemate, or enantiomer thereof:

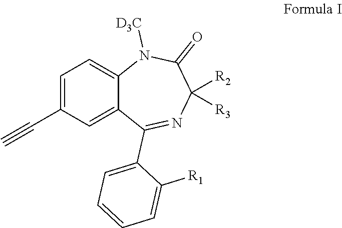

wherein: R₁ is selected from the group consisting of R₁ is selected from the group consisting of hydrogen, halo, methyl, ethyl, trideuteromethyl, trifluoromethyl, and cyclopropyl; and R₂ and R₃ are each independently selected from the group consisting of hydrogen, deuterium, tritium, and methyl.

In some embodiments, R₂ and R³ are each deuterium. In other embodiments, R₂ and R³ are each hydrogen. In other embodiments, one or both of R₂ or R₃ is methyl.

In some embodiments, the compound is selected from the compounds set forth in Table 1:

TABLE 1
Exemplary Deuterated Compounds

| | | | Chemical | Molecular | |
|---|---|---|---|---|---|
| | Name | Compound | Formula | Weight | Activity |
| Diazepam Analog | DiD₃ | 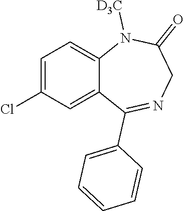 [SMILES: [2H]C([2H])([2H])N1C(=O)CN=C(c2ccccc2)c2cc(Cl)ccc21] | C₁₆H₁₀D₃ClN₂O | 287.75871 | Inactive |
| Diazepam Analog | DiD₅ | 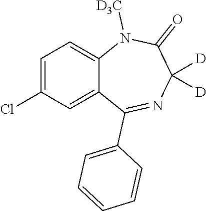 [SMILES: [2H]C1([2H])N=C(c2ccccc2)c2cc(Cl)ccc2N(C([2H])([2H])[2H])C1=O] | C₁₆H₈D₅ClN₂O | 289.77103 | Inactive |
| QH-II-66 Analog | QHD₃ | 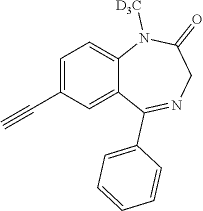 [SMILES: [2H]C([2H])([2H])N1C(=O)CN=C(c2ccccc2)c2cc(C#C)ccc21] | C₁₈H₁₁D₃N₂O | 277.33505 | Active against tumors |
| QH-II-66 Analog | QHD₅ | 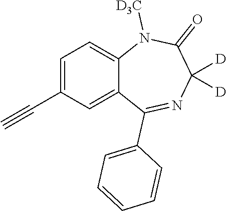 [SMILES: [2H]C1([2H])N=C(c2ccccc2)c2cc(C#C)ccc2N(C([2H])([2H])[2H])C1=O] | C₁₈H₉D₅N₂O | 279.34737 | Active against tumors |
| KRM-II- 08 Analog | KRMD₃ | 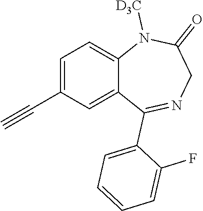 [SMILES: [2H]C([2H])([2H])N1C(=O)CN=C(c2ccccc2F)c2cc(C#C)ccc21] | C₁₈H₁₀D₃FN₂O | 295.32551 | Active against tumors |
| KRM-II- 08 Analog | KRMD₅ | 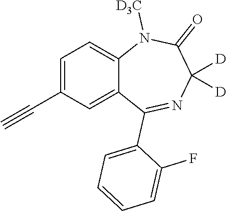 [SMILES: [2H]C1([2H])N=C(c2ccccc2F)c2cc(C#C)ccc2N(C([2H])([2H])[2H])C1=O] | C₁₈H₈D₅FN₂O | 297.33783 | Active against tumors |

In another embodiment, a pharmaceutical composition is provided, the composition comprising a compound according to Formula I, or a pharmaceutically acceptable salt, racemate, enantiomer, or derivative thereof; and at least one pharmaceutically acceptable carrier. In embodiments, the pharmaceutical compositions disclosed herein are formulated for the treatment of cancer. In specific embodiments, the Formula I compound administered to the subject is selected from the compounds set forth in Table 1.

The pharmaceutically acceptable excipient, or carrier, must be “acceptable” in the sense of being compatible with the other ingredients of the composition and not deleterious to the recipients thereof. The disclosure further includes a pharmaceutical composition, in combination with packaging material suitable for the pharmaceutical composition, including instructions for the use of the composition in the treatment of subjects in need thereof.

Pharmaceutical compositions include those suitable for enteral (e.g., oral, sublingual, buccal, or rectal), parenteral (e.g., intravenous, intramuscular, subcutaneous, intraarterial, intratumoral), intranasal, inhaled, vaginal, or transdermal administration. In a specific embodiment, the pharmaceutical compositions are formulated for intravenous administration, e.g., by injection or infusion. In another specific embodiment, the pharmaceutical compositions are formulated for oral administration.

The pharmaceutical compositions may be prepared by any methods well known in the art of pharmacy, for example, using methods such as those described in Remington: The Science and Practice of Pharmacy (21st ed., Lippincott Williams and Wilkins, 2005, see Part 5: Pharmaceutical Manufacturing). Suitable pharmaceutical carriers are well-known in the art. See, for example, Handbook of Pharmaceutical Excipients, Sixth Edition, edited by Raymond C. Rowe (2009). The skilled artisan will appreciate that certain carriers may be more desirable or suitable for certain modes of administration of an active ingredient. It is within the purview of the skilled artisan to select the appropriate carriers for a given composition.

For parenteral administration, suitable compositions include aqueous and non-aqueous sterile suspensions for intravenous administration. The compositions may be presented in unit dose or multi-dose containers, for example, sealed vials and ampoules.

For oral administration, suitable compositions include liquids, capsules, tablets, chewable tablets, soluble films, powders, and the like.

As will be understood by those of skill in this art, the specific dose level for any particular subject will depend on a variety of factors, including the activity of the agent employed; the age, body weight, general health, and sex of the individual being treated; the particular disease to be treated; the time and route of administration; the rate of excretion; and the like.

In embodiments, an effective dose of a Formula I compound according to the present disclosure may range from about 0.01 mg/kg/day to about 100 mg/kg/day, or from about 0.01 mg/kg/day to about 10 mg/kg/day, or from about 0.1 mg/kg/day to about 100 mg/kg/day, or from about 0.1 mg/kg/day to about 10 mg/kg/day, or from about 1 mg/kg/day to about 10 mg/kg/day. In embodiments, the dose of a Formula 1 compound is at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 30, 40, 50, 60, 70, 80, 90, or 100 mg/kg/day, or any selected range of values there between.

In another embodiment, a method of treating cancer in a subject in need thereof is provided, the method comprising administering to the subject an effective amount of a compound according to Formula I as disclosed herein, or a pharmaceutically acceptable salt, racemate, or enantiomer thereof.

In embodiments, the subject is a mammal. In a more specific embodiment, the subject is a human.

In embodiments, the cancer is any primary or metastatic solid tumor, including pediatric and adult tumors. In specific embodiments, the cancer is selected from the group consisting of melanoma, glioblastoma, medulloblastoma, and lung cancer. In a more specific embodiment, the lung cancer is non-small cell lung cancer (NSCLC).

In embodiments, administering comprises enteral or parenteral administration. In more specific embodiments, enteral administration comprises oral, sublingual, or buccal administration. In other specific embodiments, parenteral administration comprises intravenous, intramuscular, subcutaneous, intraarterial, or intratumoral administration. Compositions comprising Formula I compounds can be formulated for administration by any suitable enteral or parenteral administration.

In embodiments, the compound is administered at a dose of from about 0.1 mg/kg/day to about 100 mg/kg/day. In a more specific embodiment, the compound is administered at a dose of from about 1 mg/kg/day to about 30 mg/kg/day.

In embodiments, the methods disclosed herein further comprise administering to the subject one or more additional active agents. Illustratively, the one or more additional active agents are selected from the group consisting of an anti-inflammatory agent, an immunosuppressive agent, a corticosteroid, and a chemotherapeutic agent selected from the group consisting of an alkylating agent, a platinum drug, an antimetabolite, an anti-tumor antibiotic, a topoisomerase inhibitor, a mitotic inhibitor, a differentiating agent, an immune checkpoint inhibitor, and a hormone therapy.

In other embodiments, the methods disclosed herein further comprise administering radiation therapy to the subject. In embodiments, the methods disclosed herein further comprise administration of an immune checkpoint inhibitor, including but not limited to PD-1 inhibitors (e.g., pembrolizumab, nivolimumab, cemiplimab, etc.); PD-L1 inhibitors (e.g., atezolizumab, avelumab, durvalumab, etc.); CTLA-4 inhibitors (e.g., ipilimumab, tremelimumab, etc.); and LAG-3 inhibitors (e.g., relatimab, opdualag, etc.); and combinations thereof. In a specific embodiment, the checkpoint inhibitor is a PD-L1 inhibitor.

In specific embodiments, the Formula I compound administered to the subject is selected from the compounds set forth in Table 1.

### EXAMPLES

The following example is given by way of illustration is not intended to limit the scope of the disclosure.

### Example 1. Cytotoxicity Assays

Cytotoxicity of DiD3, DiD5, QHD3, QHD5, KRMD3, and KRMD5 was assessed in A375 human melanoma cells, LN18 human glioma cells, and H1792 human NSCLC cells.

Each compound powder was weighed and dissolved in DMSO to a concentration of 40 mM. Dissolved compounds were stored at 4° C. Drug dilutions were prepared in phenol free medium as follows:

| | | Volume of | |
|---|---|---|---|
| Drug Concentration | Volume of | 40 mM | Volume of |
| (μM) | medium | stock | prediluted drug |
| 30 | 2 mL | 1.5 μL | / |
| 25 | 2 mL | 1.25 μL | / |
| 20 | 2 mL | 1.0 μL | / |
| 15 | 1 mL | / | 1 mL of 30 μM |
| 10 | 1 mL | / | 1 mL of 20 μM |
| 7.5 | 1 mL | / | 1 mL of 15 μM |
| 5 | 1 mL | / | 1 mL of 10 μM |
| 2.5 | 1 mL | / | 1 mL of 5 μM |
| 1.25 | 1 mL | / | 1 mL of 2.5 μM |
| 0.625 | 1 mL | / | 1 mL of 1.25 μM |
| 0.3125 | 1 mL | / | 1 mL of 0.625 μM |
| DMSO | 2 mL | / | 1.5 μL of DMSO |

Cell culture process: Optimum cell number for each cell line was determined and the cell number giving about 1.0 OD value after one hour of incubation with the CellTiter 96® AQueous One Solution Cell Proliferation Assay reagent (Promega) was selected for cell proliferation experiments. On Day 1, cells were trypsinized, counted, and diluted to: A375 human melanoma cells: 30000 cells/mL (3000 cells/100 μL or 3000 cells/well); LN18 human glioma cells: 50000 cells/mL (5000 cells/100 μL or 5000 cells/well); and H1792 human NSCLC cells: 12000 cells/mL (1200 cells/100 μL or 1200 cells/well).

100 μL of cell suspension was added to 96 well plates. Rows B to G and columns 2 to 11 were seeded with cells. Outer wells around the plate were filled with 100 μL phosphate buffered saline (PBS), except wells A2 to A6, which were filled with medium without cells. Cells were then incubated 24 hours and allowed to attach.

On Day 2: drug and controls were added to the plated cells as follows: background control: medium in wells A2 to A6 was aspirated and replaced with 100 μL fresh medium; control and test dilutions: medium was carefully aspirated and 100 μL of DMSO control or diluted drug was added to 5 consecutive wells in the same row. After drug addition, plates were returned to the incubator for 48 to 72 hours.

On Day 5 or 6:20 μL of CellTiter 96® AQueous One Solution Cell Proliferation Assay reagent (Promega) was added to each control or test well, incubated 1 to 2 hours, and then OD was acquired at 490 nm.

Data analysis: the average OD from the five wells containing medium and no cells (A2 to A6) was calculated and the value subtracted from each individual OD reading. The OD readings for each drug dilution was normalized to DMSO control, set as 100% cell viability/proliferation. Log drug concentration versus percentage viability was then plotted using GraphPad Prism.

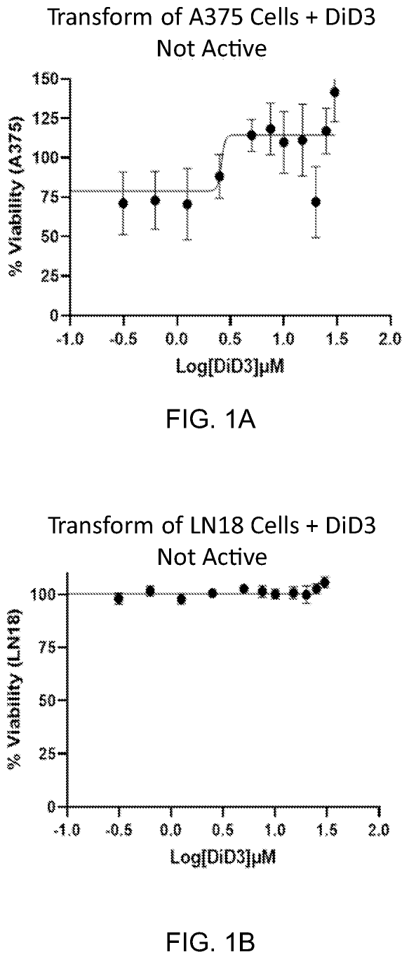

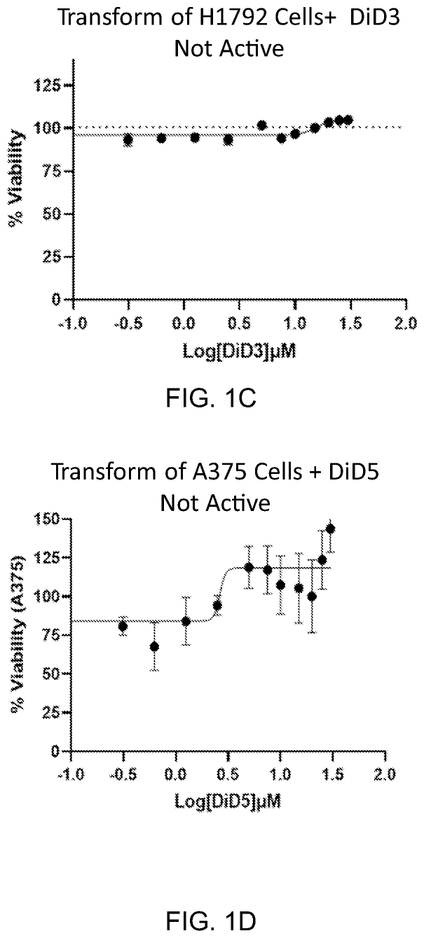

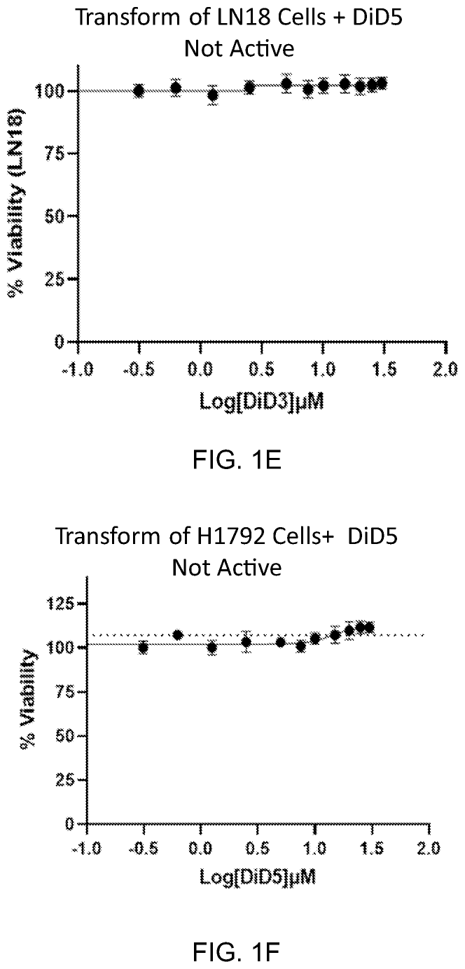

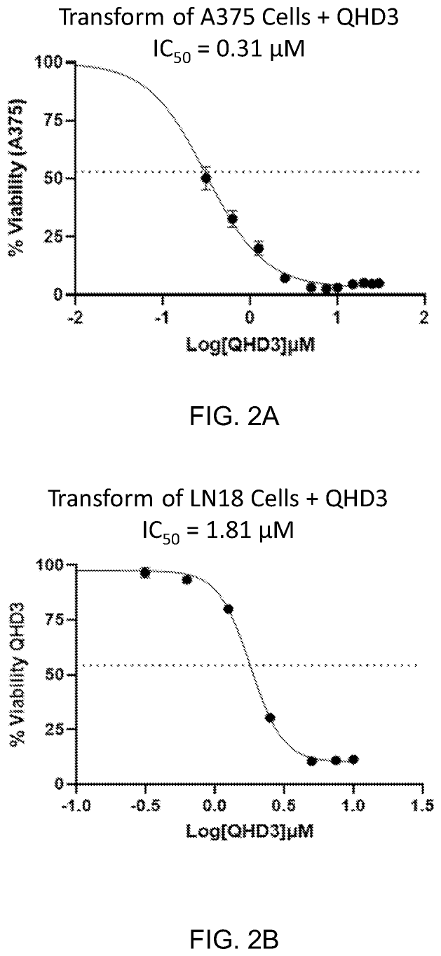

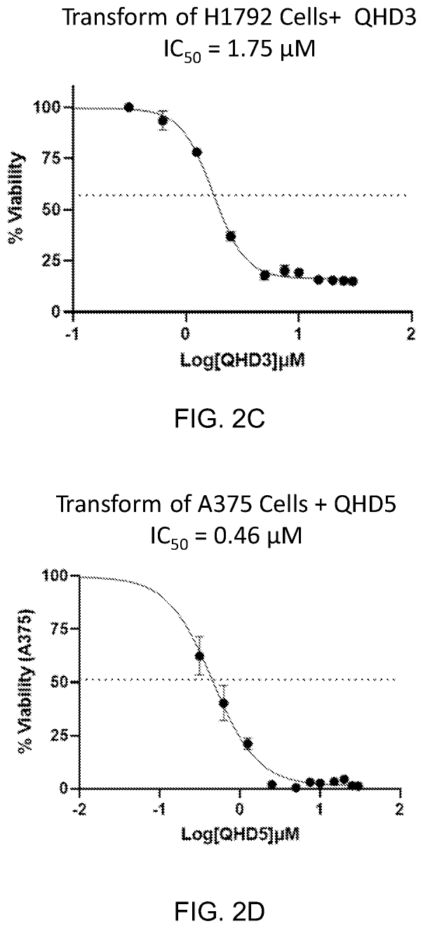

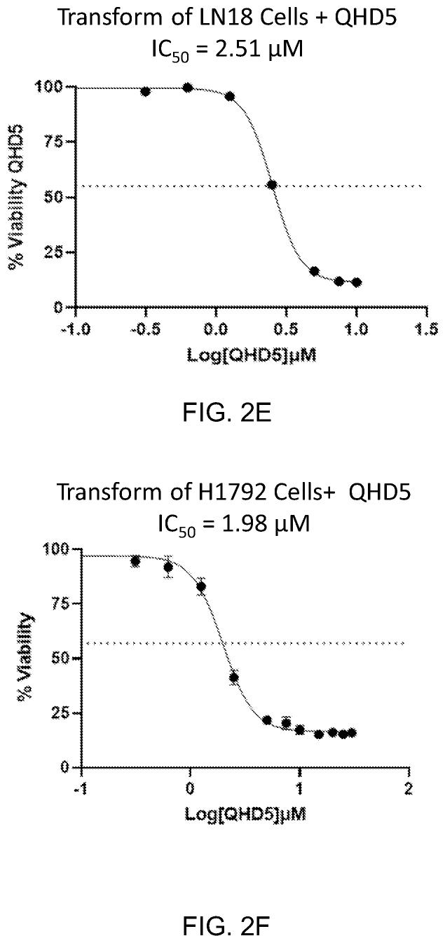

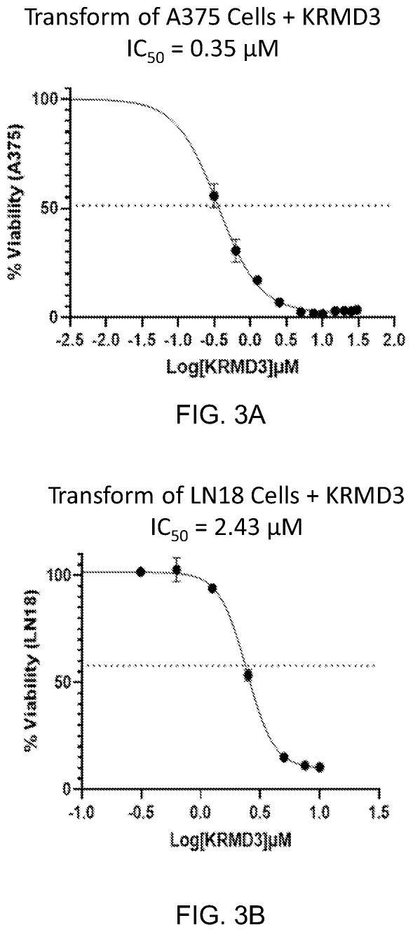

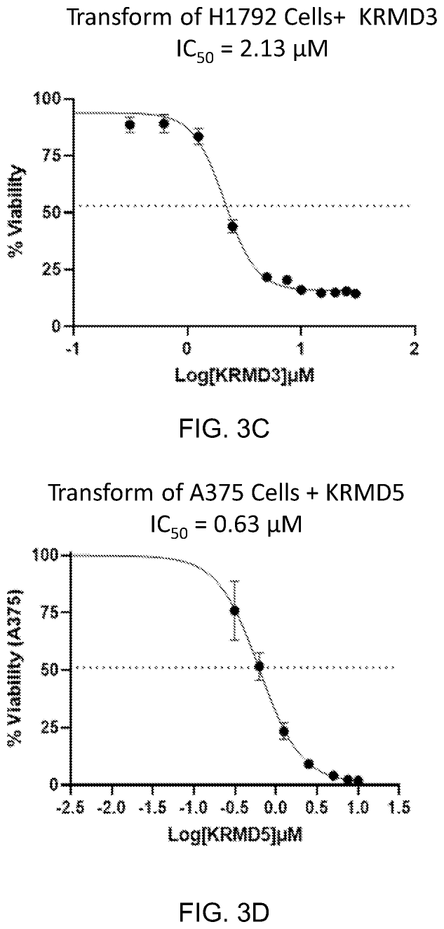

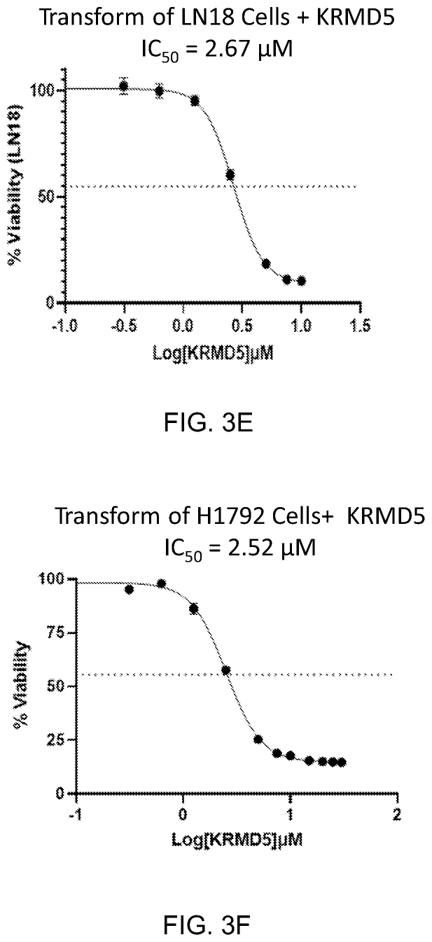

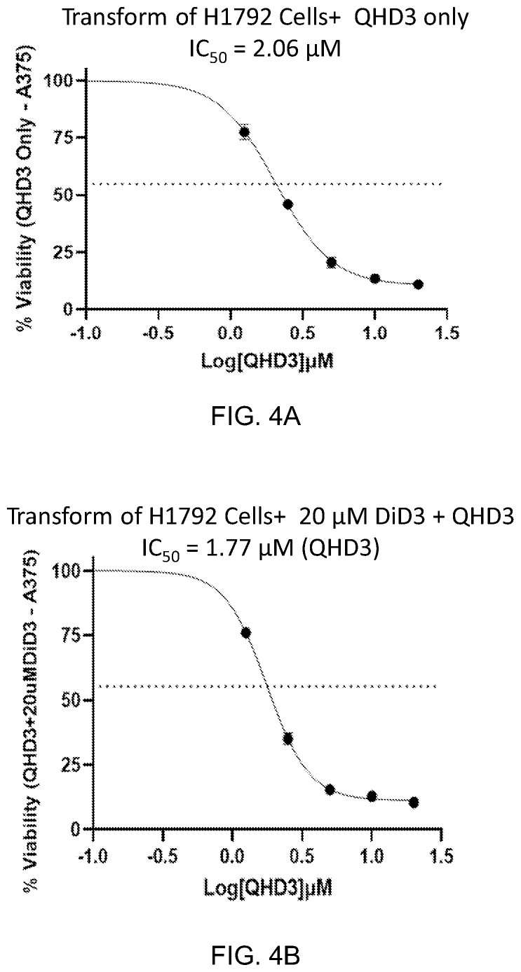

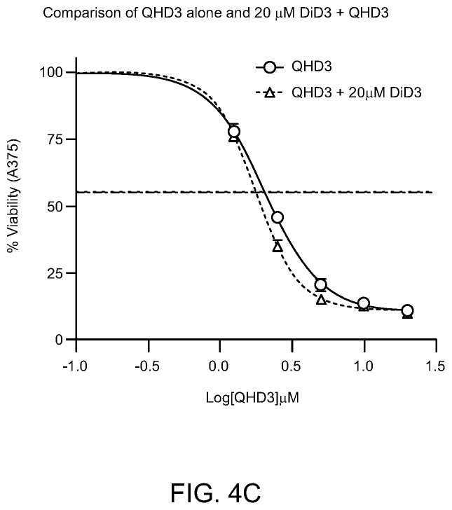

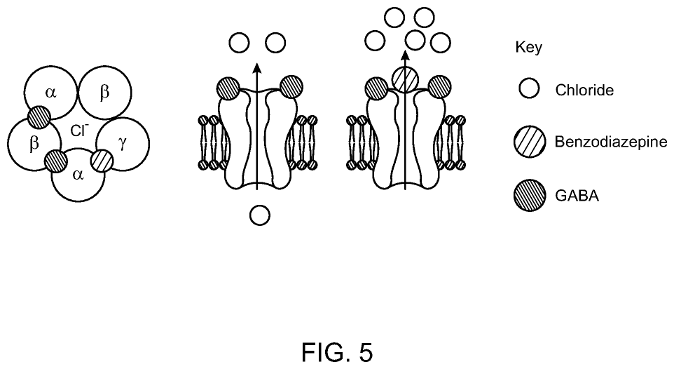

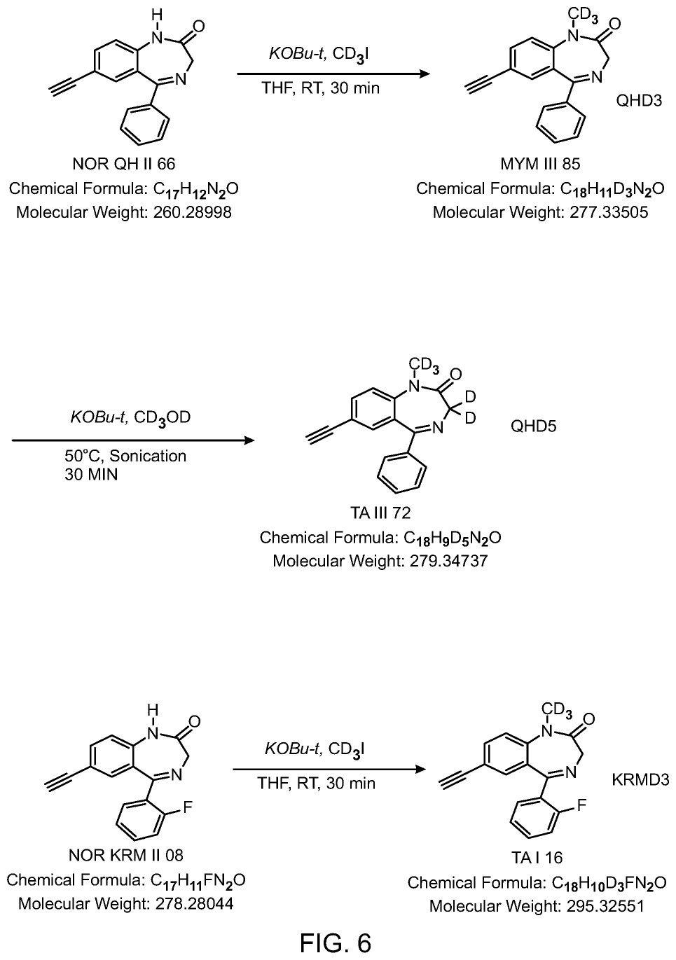

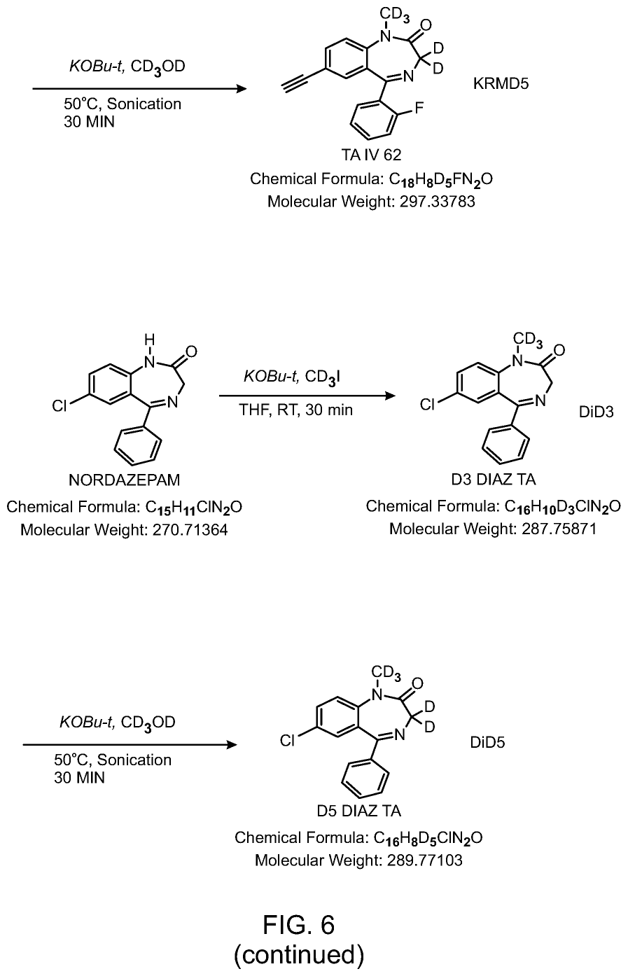

Results are shown in FIGS. 1A-3F and summarized in Table 2.

TABLE 2
Cytotoxicity of deuterated compounds (IC₅₀, μM)

| Human | | | | | | |
|---|---|---|---|---|---|---|
| patient- | | | | | | |
| derived | | | | | | |
| cell | | | | | | |
| line | DiD3 | DiD5 | QHD3 | QHD5 | KRMD3 | KRMD5 |
| A375 | Not | Not | 0.31 | 0.46 | 0.35 | 0.63 |
| | Active | Active | | | | |
| LN18 | Not | Not | 1.81 | 2.51 | 2.43 | 2.67 |
| | Active | Active | | | | |
| H1792 | Not | Not | 1.75 | 1.98 | 2.13 | 2.52 |
| | Active | Active | | | | |

Results show that modifying a benzodiazepine structure with alkynyl and deuterium moieties according to Formula I confers cytotoxicity to the chemical class, as compounds lacking the alkynyl moiety, such as DiD3 and DiD5, are non-cytotoxic against the tested human patient-derived cancer cell lines.

Aspects of the present disclosure can be described with reference to the following numbered clauses, with preferred features laid out in dependent clauses.
1. A compound according to Formula I, or a pharmaceutically acceptable salt, racemate, or enantiomer thereof:

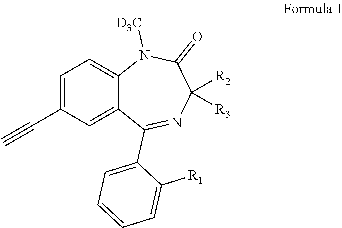

wherein:
R₁ is selected from the group consisting of hydrogen, halo, methyl, ethyl, trideuteromethyl, trifluoromethyl, and cyclopropyl; and
R₂ and R³ are each independently selected from the group consisting of hydrogen, deuterium, tritium, and methyl.
2. The compound according to clause 1, wherein R₁ is Cl, F, or Br.
3. The compound according to clause 1 or clause 2, wherein R₂ and R₃ are each hydrogen.
4. The compound according to clause 1 or clause 2, wherein R₂ and R₃ are each deuterium.
5. The compound according to clause 1, wherein the compound is selected from the group consisting of QHD3, QHD5, KRMD3, and KRMD5.
6. A pharmaceutical composition comprising:
   an effective amount of the compound according to any of claims 1-5; and
   a pharmaceutically acceptable carrier.
7. A method of treating cancer in a subject in need thereof, the method comprising administering to the subject an effective amount of a compound according to Formula I, or a pharmaceutically acceptable salt, racemate, or enantiomer thereof.
8. The method according to clause 7, wherein R₁ is Cl, F, or Br.
9. The method according to clause 7 or clause 8, wherein R₂ and R₃ are each hydrogen.
10. The method according to clause 7 or clause 8, wherein R₂ and R₃ are each deuterium.
11. The method according to any of clauses 7-10, wherein the subject is a human.
12. The method according to any of clauses 7-11, wherein the cancer is selected from the group consisting of melanoma, glioblastoma, medulloblastoma, and lung cancer.
13. The method according to any of clauses 7-12, wherein administering comprises enteral or parenteral administration.
14. The method according to any of clause 13, wherein enteral administration comprises oral, sublingual, or buccal administration.
15. The method according to clause 13, wherein parenteral administration comprises intravenous, intramuscular, subcutaneous, intraarterial, or intratumoral administration.
16. The method according to any of clauses 7-15, further comprising administering to the subject one or more additional active agents selected from the group consisting of an anti-inflammatory agent, an immunosuppressive agent, a corticosteroid, and a chemotherapeutic agent selected from the group consisting of an alkylating agent, a platinum drug, an antimetabolite, an anti-tumor antibiotic, a topoisomerase inhibitor, a mitotic inhibitor, a differentiating agent, an immune checkpoint inhibitor, and a hormone therapy.
17. The method according to any of clauses 7-16, further comprising administering radiation therapy to the subject.
18. The method according to clause 17, further comprising administering an immune checkpoint inhibitor to the subject.
19. The method according to clause 18, wherein the immune checkpoint inhibitor is a PD-L1 inhibitor.
20. The method according to any of clauses 7-19, wherein the compound is administered at a dose of from about 0.1 mg/kg/day to about 100 mg/kg/day.
21. The method according to any of clauses 7-20, wherein the compound is selected from the group consisting of QHD3, QHD5, KRMD3, and KRMD5.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

The foregoing description is illustrative of particular embodiments of the invention but is not meant to be a limitation upon the practice thereof. While particular embodiments have been illustrated and described, it would be obvious to one skilled in the art that various other changes and modifications can be made without departing from the spirit and scope of the invention. It is therefore intended to cover in the appended claims all such changes and modifications that are within the scope of this invention.

1. A compound according to Formula I, or a pharmaceutically acceptable salt, racemate, or enantiomer thereof: 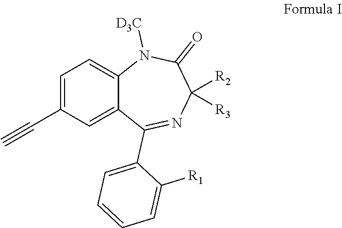
wherein:
R₁ is selected from the group consisting of hydrogen, halo, methyl, ethyl, trideuteromethyl, trifluoromethyl, and cyclopropyl; and
R₂ and R₃ are each independently selected from the group consisting of hydrogen, deuterium, tritium, and methyl.

2. The compound according to claim 1, wherein R₁ is Cl, F, or Br.

3. The compound according to claim 1, wherein R₂ and R₃ are each hydrogen.

4. The compound according to claim 1, wherein R₂ and R₃ are each deuterium.

5. The compound according to claim 1, wherein the compound is selected from the group consisting of:

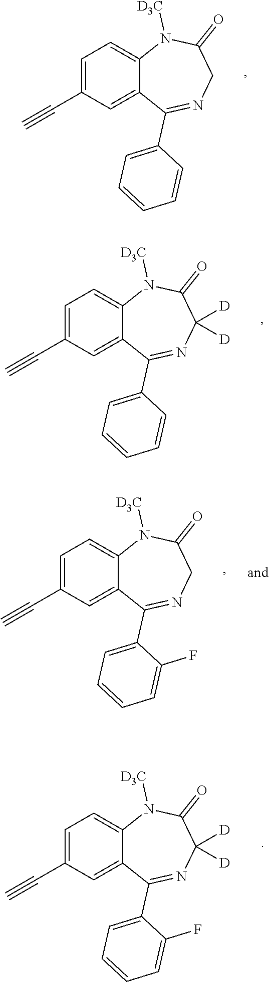

6. A pharmaceutical composition comprising:
an effective amount of the compound according to claim 1; and
a pharmaceutically acceptable carrier.

7. A method of treating cancer in a subject in need thereof, the method comprising administering to the subject an effective amount of a compound according to Formula I, or a pharmaceutically acceptable salt, racemate, or enantiomer thereof: 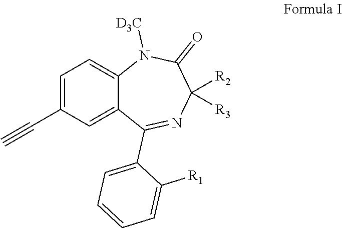
wherein:
R₁ is selected from the group consisting of hydrogen, halo, methyl, ethyl, trideuteromethyl, trifluoromethyl, and cyclopropyl; and
R₂ and R₃ are each independently selected from the group consisting of hydrogen, deuterium, tritium, and methyl.

8. The method according to claim 7, wherein R₁ is Cl, F, or Br.

9. The method according to claim 7, wherein R₂ and R₃ are each hydrogen.

10. The method according to claim 7, wherein R₂ and R₃ are each deuterium.

11. The method according to claim 7, wherein the subject is a human.

12. The method according to claim 7, wherein the cancer is selected from the group consisting of melanoma, glioblastoma, medulloblastoma, and lung cancer.

13. The method according to claim 7, wherein administering comprises enteral or parenteral administration.

14. The method according to claim 13, wherein enteral administration comprises oral, sublingual, or buccal administration.

15. The method according to claim 13, wherein parenteral administration comprises intravenous, intramuscular, subcutaneous, intraarterial, or intratumoral administration.

16. The method according to claim 7, further comprising administering to the subject one or more additional active agents selected from the group consisting of an anti-inflammatory agent, an immunosuppressive agent, a corticosteroid, and a chemotherapeutic agent selected from the group consisting of an alkylating agent, a platinum drug, an antimetabolite, an anti-tumor antibiotic, a topoisomerase inhibitor, a mitotic inhibitor, a differentiating agent, an immune checkpoint inhibitor, and a hormone therapy.

17. The method according to claim 7, further comprising administering radiation therapy to the subject.

18. The method according to claim 7, further comprising administering an immune checkpoint inhibitor to the subject.

19. The method according to claim 18, wherein the immune checkpoint inhibitor is a PD-L1 inhibitor.

20. The method according to claim 7, wherein the compound is administered at a dose of from about 0.1 mg/kg/day to about 100 mg/kg/day.

21. The method according to claim 7, wherein the compound is selected from the group consisting of: [chemical structure]
